(19) United States
(12) Patent Application Publication
(10) Pub. No.: US 2023/0263660 A1
(43) Pub. Date: Aug. 24, 2023

(54) INSTRUMENT FOR THE SURGICAL TREATMENT OF CATARACT, AIMED AT EXTRACTING THE NUCLEUS OF THE CRYSTALLINE LENS

(71) Applicant: STUDIO OCULISTICO DR.SSA ANTONIETTA CARBONE S.N.C., SIRACUSA (IT)

(72) Inventor: FERDINANDO MUNNO, SIRACUSA (IT)

(21) Appl. No.: 18/012,686

(22) PCT Filed: Jun. 21, 2021
(86) PCT No.: PCT/IT2021/050192

§ 371 Date: Dec. 23, 2022

(30) Foreign Application Priority Data
Jun. 24, 2020 (IT) 102020000015139

(51) Int. Cl. A61F 9/007

(52) U.S. Cl. CPC A61F 9/00736; A61B 2017/00358

(57) ABSTRACT

This invention concerns the field of ophthalmic surgical instruments used in cataract operations. The invention consists, in particular, to a surgical instrument designed to incorporate the nucleus’s crystalline lens and extract it entirely off the eye. The found consists of a tubular handle rotatable, that operates to an insertion mechanism, to which is connected to an intermediate element from which they take place, through two channels: a cable together joined a collection chamber, adapted to incorporate the nucleus’s crystalline lens; a cursor, suitable for the hermetic closure of the collection chamber and a fixing device, used to weaken the internal fibers of the nucleus. The found allows the elimination of the phase of fragmentation and aspiration of the nucleus’s crystalline lens with the integral removal of the nucleus avoiding micro-injuries or damage to the capsular bag used by ultrasound in the fragmentation phase and the consequent serious intraoperative complications.

### TECHNICAL FIELD

The present invention relates to ophthalmic surgical instruments and in particular to those used in cataract surgery.

### BACKGROUND ART

As known, the cataracts is a typical eye disease of the elderly which consists in the opacification of the crystalline lens, which causes a progressive reduction of the visual capacity up to blindness.

The most frequent cause of cataract is aging, but it can also derive from eye trauma, ophthalmic or systemic diseases, hereditary or congenital defects. In the first case, more widespread, we speak of senile cataract caused by the hardening and slow yellowing of the fibers of the crystalline.

The cataract removal operation consists in the removal of the nucleus of the opacified crystalline and its subsequent replacement with a special artificial lens, suitable for the patient’s characteristics.

Currently, this operation is carried out according to a technique called “Facoemulsification”, through which, at first, the membrane that surrounds the crystalline, i.e. capsular bag, is opened, then the crystalline nucleus is fragmented using an ultrasonic probe, and finally fragments of the nucleus and cortical masses of the opacified crystalline are sucked in.

The technique involves an incision of about 2 mm on the cornea (in the anterior part of the eye), the opening of the capsular bag through special pliers (capsuloressi), the detachment of the nucleus from its envelope through the input of water into the bag (hydrodissection). The procedure is completed with the fragmentation of the nucleus with a special instrument, the facoemulsificator, throughthe use of ultrasound and its subsequent aspiration. Finally, a particular intraocular lens is implanted that also corrects the visual defect of the patient, graduated and studied for each individual patient.

The greatest risk of the intervention executed with the current technique is the lesion or irreversible damage of the micrometric structures of the capsular bag that may occur during the phase of fragmentation of the nucleus’s crystalline lens inside it.

In this phase, in fact, the ultrasound emitted by the phaeomulsificator can cause the rupture of the posterior capsule of the crystalline lens, formed by a micrometric structure just 4 microns thick (a human hair measures about 10 times more than the crystalline capsule).

Complications related to the posterior capsule rupture consist in the fall of nucleus fragments into the vitreous chamber, that is the space between the posterior surface of the crystalline and the retina.

These unpleasant complications, besides compromising the success of the operation, do not allow the regular positioning of the artificial lens in its intended location (capsular bag) and must be implanted in alternative locations with risks of falling of very small parts into the vitreous chamber of the eye. All this can involve a risk for the regular recovery of vision, a risk of inaccuracy in the calculation of artificial lens to be implanted, post-operative blurring up to chronic inflammation. The fall of fragments of the nucleus of the original crystalline lens, moreover, makes necessary and urgent a new intervention of removal of said fragments through the so-called “vitrectomy”, with new risks for the retina and for the general health of the eye. Another possible complication attributable to the current technique is the incomplete extraction of the crystalline lens that is achieved following the failure to recover a microfragment of the crystalline lens by the aspirator because the microfragment appears to have hidden behind elements anatomical that could be damaged by suction and suction. Also in this case, a subsequent intervention must be carried out to remove the microfragment.

In rarer cases, finally, corneal burns may occur due to the lack of cooling of the phaeomulsificator in the fragmentation phase. You have also consider the medical-legal problems and disputes concerning intra-operative eye damage essentially linked to the consequences of the rupture of the posterior capsule of crystalline.

On the market there are currently alternative techniques, especially used in Oriental Countries, where the availability of equipment that exploit ultrasound are limited. These techniques, however, provide the opening of the eye with clean cuts of more than 3 cm and their subsequent sutures to close them. These are extremely bloody interventions for the eye, also characterized by the onset of many complications. In this type of surgery a scleral incision is made on the eye and then a lesion of the crystalline capsule, which is removed in block without ultrasound with dedicated instruments. The drawback is that it is made a cut of several centimeters, with subsequent suture of the eye and with slower shooting times.

### DISCLOSURE OF INVENTION

The invention in question, according to the claims, would introduce and allow a new technique in the ophthalmological field based on the complete removal of the nucleus of the crystalline lens instead of the current technique of fragmentation of the nucleus’s crystalline lens.

The surgical instrument, according to the found, introduces a technique that does not deviate from the current one in the early stages, as it will always proceed to the incision of the cornea, to the opening of the capsular bag through the special plier and finally to the hydrodissection of the nucleus. In this step, however, we will not proceed to the fragmentation and aspiration by the facoemulsificator, but removing the nucleus by the surgical instrument object of the present invention.

The surgical instrument, according to the found, is inserted inside the eye through the incisions made during the first phases of the surgery, thus allowing to arrive the height of the capsular bag. At this point, through an insertion mechanism that is operated by the handle, a bio-compatible collection chamber emerges, which will wrap the entire nucleus of the crystalline lens, leaving the capsular bag unharmed, through a circular gripping mechanism. The whole movement will not generate any pressure inside the eye but will be part of a regular surgical process, facilitated in the maneuvers by the introduction of a viscoelastic substance.

Through of a circular grip movement, in fact, the nucleus will be wrapped in the collection chamber of the instrument that is intended to be patented. Through the incorporation of the nucleus in the collection chamber of the surgical instrument is obtained the subsequent removal of the same without the use of the facoemulsificator. This avoids the use of ultrasound and the onset of countless related complications. Then proceed to the closing of the collection chamber with hermetic closing cursor. In this phase we proceed with the development of the fixing device inside the collection chamber with the aim of weakening the internal fibers of the crystalline lens. The surgical maneuver ends with the withdrawal of the collection chamber and the fixing device in its initial housing, removing the crystalline lens without fragmenting it as a result.

The surgery always ends, as expected by the current technique, with the implantation of an intraocular lens in the capsular bag.

The mechanism of this invention could be assimilated to that currently used in the technique of abdominal laparoscopy for the removal of the gallbladder: through this technique it is possible to remove large organs through incisions on the abdomen of just 2 cm. The organs, once freed from the vascular and nervous constraints, are placed in a special “pocket”, sealed and removed from the organism.

In the same way, the surgical instrument according to the found aims to incorporate the nucleus of the crystalline lens after the detachment of the capsular bag from its involucre (then, after the phase of hydrodissection), and to remove it through the incisions practiced on the cornea.

The aim of this instrument is to reduce definitively the incidence of intraoperative complications related to cataract intervention by eliminating, in fact, the use of the facoemulsificator, a very expensive instrument.

In particular, the invention aims to eliminate the phase of fragmentation of the nucleus’s crystalline lens inside the eye, avoiding the risk of the occurrence of the aforementioned complications and allowing the definitive removal of the nucleus of the crystalline lens, since this an instrument designed to incorporate the nucleus of the crystalline lens and extract it intact from the eye. It therefore avoids the use of ultrasound and the onset of countless related complications, such as micro-lesions or damage produced to the capsular bag in the phase of fragmentation.

This pourpose is achieved by the surgical instrument according to the first claim attached.

Another purpose of this invention is to design a surgical tool which, in addition to meeting the above purpose, can be easily sterilized after being used on a patient, so that it can be used for another patient.

Other advantageous characteristics are subject of the alleged claims, which are considered an integral part of this text.

The aforementioned purposes are achieved through the present found, in accordance with the claim n.1, which has an approximate weight of about 30 grams and is composed of:
a rotating plastic handle of tubular shape that operates an insertion mechanism;
an intermediate element, that is an elongated body of tubular shape with open sections, joined to one end of handle end and equipped with two channels inside it, designed to contain respectively:
   a nitinol cable (or other deformable biocompatible metal alloy) ;
   a fixing device, used to weaken the nucleus’s internal fibres;
an collection chamber of oval shape, made of silicone material;
a cursor adapted for hermetically closing of the collection chamber.

### BRIEF DESCRIPTION OF DRAWINGS

Further characteristics and advantages of the invention will be more evident by means the detailed description of a preferred but not exclusive form of use, illustrated as a non-exhaustive example with reference to the attached drawings in which the figures show:

FIG. 1: the whole instrument in prospectus, in view from above, in which are distinguished: the handle, the intermediate element and the cable;

FIG. 2: the intermediate element of the instrument, in view the exploded axonometric, in which are distinguished: the fixing device, cable and collection chamber;

FIG. 3: a portion of the intermediate element of the instrument in longitudinal section in which are distinguished: the cable, the collection chamber, the cursor and the fixing device when the instrument is in closed mode.

FIG. 4: a portion of the intermediate element of the instrument in view from above, when the instrument is in closed mode;

FIG. 5: a portion of the intermediate element of the instrument in longitudinal section, during the withdrawal phase of the cable and of the collection chamber;

FIG. 6: a portion of the intermediate element of the instrument in view from above, during the withdrawal phase of the cable and the collection chamber;

FIG. 7: a portion of the intermediate element of the instrument in longitudinal section, during the sliding phase of the cursor along the two branches of the cable;

FIG. 8: a portion of the intermediate element of the instrument in view from above, during the sliding phase of the cursor along the two branches of the cable; Please note: the fixing device is still inside the intermediate element;

FIG. 9: a portion of the intermediate element of the instrument in longitudinal section, in the phase of progressive development of the fixing device;

FIG. 10: a portion of the intermediate element of the instrument in view from above, in the phase of progressive development of the fixing device inside the collection chamber;

FIG. 11: a portion of the intermediate element of the instrument in longitudinal section, during the withdrawal of the fixing device and subsequent withdrawal of the cable, the cursor and the collection chamber inside the intermediate element;

FIG. 12: a portion of the intermediate element of the instrument in view from above, during the withdrawal of the fixing device and subsequent return of the cable, the cursor and the collection chamber within the intermediate element;

FIG. 13: a cross section of the intermediate element of the instrument, in which are distinguished the cable, the cursor and the fixing device ;

FIG. 14: a detailed view of the cursor, precisely: in prospectus and in cross section;

FIG. 15: a detailed view of the cable, when the instrument is in closed mode, precisely: in prospectus and in cross section.

FIG. 16: a detailed view of the fixing device, precisely: in prospectus and in cross section.

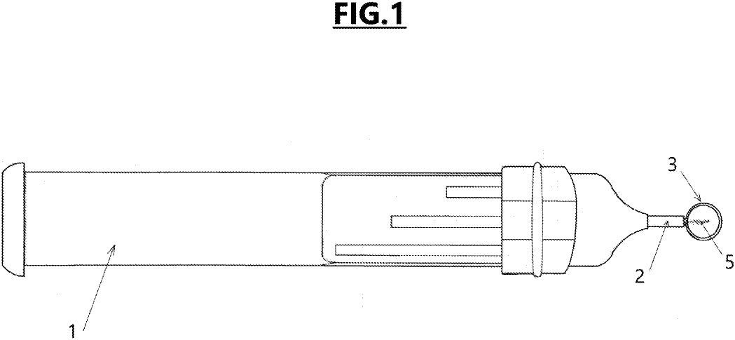

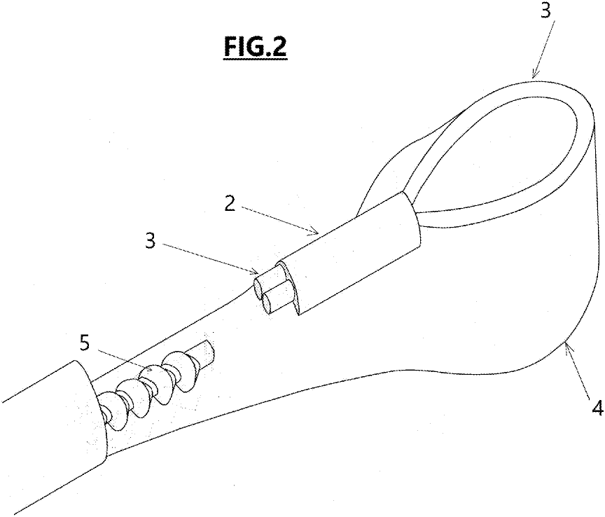

### DESCRIPTION OF AT LEAST ONE WAY OF CARRYNG OUT THE INVENTION

The present invention will now be described by way of example and not limiting, according to a preferred form of implementation, also referring to the described figures that show the instrument in question in a progressive sequence. The purpose, as exposed, is to insert the instrument into the inner cavity of the eye, more precisely in the anterior chamber through an incision of 2.75 mm. A viscoelastic substance is then inserted with the effect of filling the spaces previously occupied by the aqueous mood.

We proceed first with the capsuloressi, that is opening of the anterior capsule of the crystalline lens with dedicated plier and then with hydrodissection, that is insertion of water between the capsule of the crystalline lens and its nucleus in order to unstick the two structures.

Now proceed with the insertion in the eye of the instrument according to the found (FIGS. 3-4).

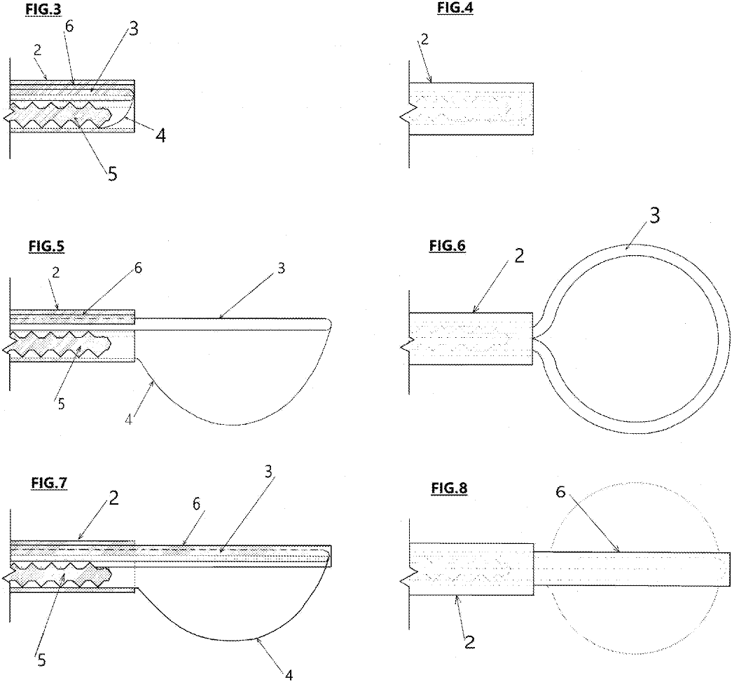

With a rotation maneuver of the handle 1 we obtain the development along the intermediate element 2 of cable 3 and, therefore, the escape of the collection chamber 4 and subsequent maneuver of collection and circular grip of the nucleus’s crystalline lens, carried out by the surgeon (FIGS. 5-6). This maneuver would be facilitated by the use of a viscoelastic substance. Then the surgeon, following the insertion of the nucleus’s crystalline lens inside the collection chamber 4 of the instrument, proceeds to the hermetic closure of the collection chamber 4 by sliding the closing cursor 6 along the branches of cable 3 (FIGS. 7-8).

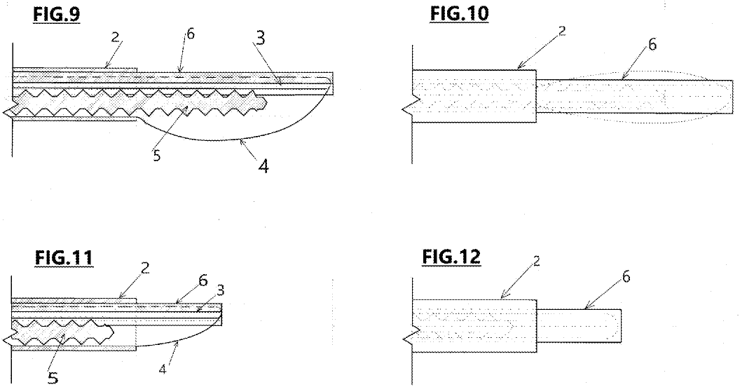

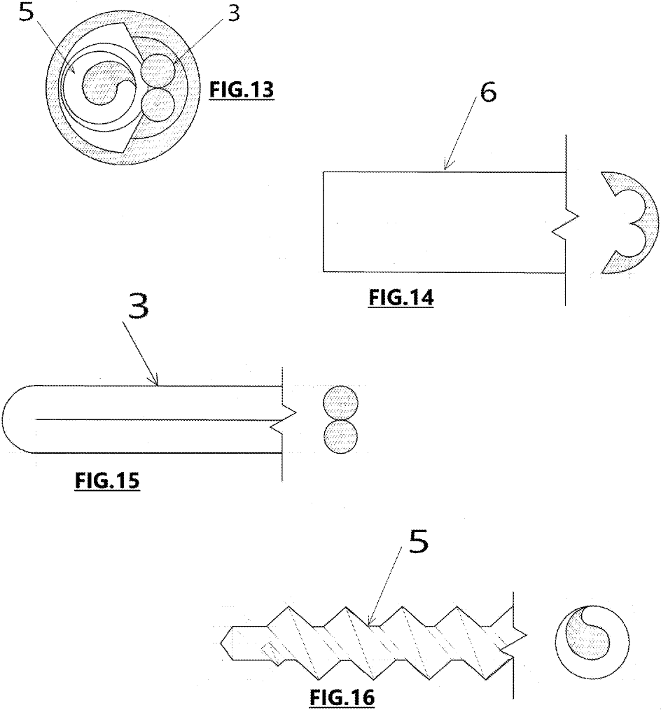

Through of the rotation mechanism of the handle 1, it is possible to obtain a progressive development of the fixing device 5, which weakens the fibres of the nucleus inside the collection chamber 4 (FIGS. 9-10).

With an opposite rotation maneuver of handle 1 is obtained the withdrawl of the fixing device 5 inside the intermediate element 2, and hermetic closing with cursor 6 of the collection chamber 4 and contextual folding of the collection chamber 4 inside of the intermediate element 2 (FIGS. 11-12).

At this point the instrument can be extracted from the eye.

From what is described it is evident that the instrument according to the invention achieves the predetermined aims.

The object of the invention is susceptible to numerous modifications and variants of a practical-applicative nature, all falling within the inventive concept expressed in the claims alleged. All the details may be replaced by other technically equivalent elements, and the materials may be different according to the needs, without leaving the scope of protection of this invention.

Although the object has been described with particular reference to the attached figures, the reference numbers used in the description and claims are used to improve the intelligence of the invention and do not constitute any limitation to the claimed scope of protection.

1. An instrument for ophthalmic surgery, adapted to remove the nucleus of a crystalline lens of an eye, in its integrity, without the use of ultrasound and without fragmentation thereof, the instrument comprising:
a tubular handle, which can be rotated in both directions to control an insertion mechanism acting on movable members of the instrument, respectively,
a deformable metal alloy cable, folded to U,
an oval-shaped collection chamber of biocompatible material with an open section,
a cursor that surrounds at least part of the branches of the cable,
a fixing device comprising at least one screw, in which said movable members are contained in an immovable intermediate element, also tubular shaped with open sections, wherein,
said cable, operated by said rotating handle, allows the output of said collection chamber adapted to wrap the nucleus of the crystalline lens,
the sliding of said fixing device, operated by said rotating handle, is intended to weaken the internal fibres of the nucleus of the eye crystalline lens after being incorporated in the collection chamber,
the intermediate element is equipped with at least two channels, adapted for receiving therein the metal alloy cable, joined to the collection chamber-(4), in the upper channel and said fixing device in the lower channel,
said cursor, operated by the rotating handle is intended to slide along said branches of the cable, allowing the sealing of said collection chamber, and
said rotating handle operated in the reverse anti-clockwise allows the withdrawal of the movable members.

2. The instrument, according to claim 1, wherein said handle operates the insertion mechanism.

3. The instrument, according to claim 1, wherein said intermediate tubular element is joined to the end of the handle.

4. The instrument according to claim 1, wherein said metal alloy cable and said collection chamber move in dependence to the rotations of the handle, to assume one of two different configurations: a first rest configuration, where said branches of the the cable, abutting each other, are arranged entirely within said intermediate element, and a second operational configuration in which said branches of the cable escaping from said intermediate element envelope the edges of the inlet section of the collection chamber-(4).

5. The instrument according to claim 1, wherein said collection chamber joined to said cable, has at least two different configurations: a first rest configuration in which the collection chamber is folded inside said upper channel of the intermediate element and a second operating configuration in which the collection chamber comes out, allowing the said open section to wrap and to incorporate the nucleus of the crystalline lens.

6. The instrument, according to claim 1, wherein the fixing device, comprising at least one screw, is dependent on rotations of the handle and has two different configurations: a first rest configuration in which the fixing device is located within said lower channel of the said intermediate tubular element and a second operating configuration in which the fixing device comes out of said intermediate element, sliding inside the collection chamber.

7. The instrument, according to claim 1, wherein the cursor has two different configurations: a first rest configuration in which the cursor houses said branches of the cable inside the upper channel of said intermediate tubular element, and a second operating configuration in which the cursor slides along said branches of the cable and cooperates with them in such a way as to seal the collection chamber.

8. The instrument according to claim 1, wherein said insertion mechanism allows at least four distinct and progressive rotation steps: a first rotation step that allows the cable joined to the collection chamber to come out of said intermediate element; a second rotation step in which the cursor comes out of said intermediate element by sliding along said branches of the cable, a third rotation step that allows the fixing device to come out of said intermediate element; and a fourth step of opposite rotation that withdraws said movable members within the intermediate tubular element, returning to a rest configuration.

9. The instrument according to claim 2, wherein said insertion mechanism allows at least four distinct and progressive rotation steps: a first rotation step that allows the cable joined to the collection chamber to come out of said intermediate element; a second rotation step in which the cursor comes out of said intermediate element by sliding along said branches of the cable, a third rotation step that allows the fixing device to come out of said intermediate element; and a fourth step of opposite rotation that withdraws said movable members within the intermediate tubular element, returning to a rest configuration.

10. The instrument according to claim 3, wherein said insertion mechanism allows at least four distinct and progressive rotation steps: a first rotation step that allows the cable joined to the collection chamber to come out of said intermediate element; a second rotation step in which the cursor comes out of said intermediate element by sliding along said branches of the cable, a third rotation step that allows the fixing device to come out of said intermediate element; and a fourth step of opposite rotation that withdraws said movable members within the intermediate tubular element, returning to a rest configuration.

11. The instrument according to claim 4, wherein said insertion mechanism allows at least four distinct and progressive rotation steps: a first rotation step that allows the cable joined to the collection chamber to come out of said intermediate element; a second rotation step in which the cursor comes out of said intermediate element by sliding along said branches of the cable, a third rotation step that allows the fixing device to come out of said intermediate element; and a fourth step of opposite rotation that withdraws said movable members within the intermediate tubular element, returning to a rest configuration.

12. The instrument according to claim 5, wherein said insertion mechanism allows at least four distinct and progressive rotation steps: a first rotation step that allows the cable joined to the collection chamber to come out of said intermediate element; a second rotation step in which the cursor comes out of said intermediate element by sliding along said branches of the cable, a third rotation step that allows the fixing device to come out of said intermediate element; and a fourth step of opposite rotation that withdraws said movable members within the intermediate tubular element, returning to a rest configuration.

13. The instrument according to claim 6, wherein said insertion mechanism allows at least four distinct and progressive rotation steps: a first rotation step that allows the cable joined to the collection chamber to come out of said intermediate element; a second rotation step in which the cursor comes out of said intermediate element by sliding along said branches of the cable, a third rotation step that allows the fixing device to come out of said intermediate element; and a fourth step of opposite rotation that withdraws said movable members within the intermediate tubular element, returning to a rest configuration.

14. The instrument according to claim 7, wherein said insertion mechanism allows at least four distinct and progressive rotation steps: a first rotation step that allows the cable joined to the collection chamber to come out of said intermediate element; a second rotation step in which the cursor comes out of said intermediate element by sliding along said branches of the cable, a third rotation step that allows the fixing device to come out of said intermediate element; and a fourth step of opposite rotation that withdraws said movable members within the intermediate tubular element, returning to a rest configuration.
